(19) United States
(12) Patent Application Publication
(10) Pub. No.: US 2026/0108672 A1
(43) Pub. Date: Apr. 23, 2026

(54) NOVEL ENHANCED PATCH PUMP SYSTEMS

(71) Applicant: Beta Bionics, Inc., Irvine, CA (US)

(72) Inventors: Michael Rosinko, Peoria, AZ (US); Mike Mensigner, San Diego, CA (US); David Lim, Irvine, CA (US); Himanshu Patel, Concord, MA (US); Patrick Castenada, Escondido, CA (US); Ryan Schoonmaker, Escondido, CA (US)

(73) Assignee: Beta Bionics, Inc., Irvine, CA (US)

(21) Appl. No.: 19/358,630

(22) Filed: Oct. 15, 2025

Related U.S. Application Data
(60) Provisional application No. 63/708,333, filed on Oct. 17, 2024.

(51) Int. Cl. A61M 5/142; A61M 5/14; A61M 5/145; A61M 5/168; A61M 5/315

(52) U.S. Cl. CPC A61M 5/14248; A61M 5/14216; A61M 5/1452; A61M 5/16804; A61M 5/31501; A61M 5/31578; A61M 2005/1402; A61M 2005/14268; A61M 2005/31588; A61M 2202/0007; A61M 2202/04; A61M 2205/502

(57) ABSTRACT

Patch pump configurations and combinations with modular aspects of dosing, storage and delivery processing of medicaments optimizes pairing and updating of the correct algorithms with minimized cognitive burdens—likewise offered for consideration methods for administering medicament from a medical device including snap together, prime with body weight and be connected—with no further steps.

### CROSS-REFERENCE TO RELATED APPLICATIONS

This document claims the benefit of priority to U.S. Provisional Application Ser. No. 63/708,333, filed Oct. 17, 2024, which is hereby incorporated by reference in its entirety.

### BACKGROUND OF THE ART

As industry-based Patent Activity has increased, the value of mechanical base configurations and the initial waves of patch pump technology have come off patent and/or opened-up new spaces for those focused on the higher end of algorithm optimization to drive such modular systems improvements, along with communications between modules being emphasized.

To these ends, even with different pumps (electro-osmotic v. SMAlloys) EOFlow was found to infringe at least one claim of Insulet's U.S. Pat. Nos. 11,299,741; 10,420,883 and 9,402,950 via ‘clutch mechanisms’ for advancing lead screws and plungers inside of a patch pump (U. S Fed District Court. D. Mass Aug. 3, 2023). Other aspects of the OMNIPOD brand of patch pump including a drive system with a walking-man hook, a clutch/tube nut for rod engagement and rachet gears with 50 teeth per wheel and their insulin reservoir with plunger and drive rod were also implicated. EP Patent 1,874,390's actuator mechanism was also successfully enforced against EOFlow in Germany (Dusseldorf Regional Court), as was their “sensor for linear detection of a lead screw, wherein prevention of rotations of leadscrew is prevented” under EP Patent 2,438,957 against Medtrum. Degrees of design freedom imparted by knowledge of the patent landscape have increased as the half dozen companies competing in this space has increased, leading up to the instant inventions for novel enhanced patch pump systems.

User preference and delivery of medicaments drive the optimization of many technologies involved in treating diabetes with infusion pumps.

Often slowing down the acceptance of better systems are the impediments overcome by the prior failed attempts. For infusion pumps the evolution has proceeded in reverse. First to patient were SYSTEMS FOCUSED ON HIGH LEVELS OF COGNITIVE ENGAGEMENT—now understood to be a detriment or not needed to achieve best blood sugar results.

A new paradigm, it is respectfully proposed, takes the opposite approach. Beta Bionics have discovered that closed-loop systems for treating diabetes are manifested in patch pumps both in combination with extant BETA BIONICS systems (as referenced above) and in combination with third party insulin delivery methods. In essence, there a several principled approaches comprising combinations of novel drive train concepts with glucagon pumps which respond to glucose level control signals, inter alia.

For example, the Beta Bionics Bihormonal system using the iLET App for the Bionic Pancreas Patch Pump, and standalone systems working as accessories to third party insulin delivery systems are disclosed here and claimed below.

The principled basis for lower cognitive load for users and modular systems which require little or no user inputs have become an industry standard. For example, the ILET ® BIONIC PANCREAS® with only the body weight of a user to prime said device delivers insulin as accurately as any system on the market with no keystrokes required.

### OBJECTS AND SUMMARY OF THE INVENTIONS

Briefly stated, any modular system aspect claimed and disclosed above in the list of pending patents is incorporated expressly by reference, and may be implemented, in whole or in part as the instant inventions or patch pumps, being the equivalent of any said patents and disclosures from the iLET brand of BIONIC PANCREAS system to any bihormonal disclosures, including all needed software and algorithm interfacing to manage blood sugar control in identity with the other invention, combinations and subcombinations thereof.

According to embodiments of the instant disclosures, there are provided Infusion Schemata each operatively Linked and novelly configured according to then present invention with at least one of those systems re-designed to impart maximum user control with minimum input. Offered for consideration are at least about the following of which the instant system are comprised of—alone or in combination
Dual cannula (“snakebite”)
Coaxial cannula
Top/bottom cannula
Micro-needle arrays.
   Bihormonal Glucagon patch control schemas and implementations in several different forms, namely:
As part of the Beta Bionics Bihormonal system.
As part of a standalone system that as an accessory to 3^{rd} party insulin delivery systems.
Drivetrain concepts
Single motor to drive two medicaments, one device (clutch, ratchet)
Nitinol wire approach to the same.

### BRIEF DESCRIPTION OF THE DRAWINGS

The systems, methods, and devices of this disclosure each have more than several innovative aspects, no single one of which is solely responsible for all of the desirable attributes disclosed herein. Details of one or more implementations of the subject matter described in this specification are set forth in the accompanying drawings and the description below, which are merely illustrative and not limiting, often omitting state of the art details for clarity of description, as known to those having a modicum of skill in the art.

FIG. 1 shows a cartooned schematic of an embodiment of a patch pump functioning as part of a patch pump system or kit and component of a third party insulin delivery system driven by the BETA BIONICS software and logic structures as listed in patents incorporated expressly by reference herein infra, including each proprietary elements of said data delivered by patch pumps, according to the present inventions.

FIG. 2 shows a cartooned schematic of an example blood glucose control system embodied in present inventions.

FIG. 3 shows a cartooned schematic of an example blood glucose control system embodied in present inventions.

FIG. 4 shows a cartooned schematic of an example blood glucose control system embodied in in present inventions.

FIG. 5 shows a cartooned schematic of an example blood glucose control system embodied in a in present inventions.

FIG. 6 shows a cartooned schematic of an example blood glucose control system embodied in in present inventions.

FIG. 7 shows a cartooned schematic of an example blood glucose control system

FIG. 8 shows a cartooned schematic of an example blood glucose control system embodied in present inventions.

FIG. 9 shows a cartooned schematic of an example blood glucose control system embodied in a in present inventions.

FIG. 10 illustrates a partial plan view of an example ambulatory medical device being a novel enhanced Patch Pump system (PPs).

FIG. 11 illustrates a dorsal view showing sections removable for a combination of durable and disposable assembly view of the ambulatory medical device shown manifested in a novel enhanced patch pump configuration.

Ambulatory medical devices allow patients the freedom to treat themselves while being mobile. The patient may be tasked with operating the ambulatory medical device. In some cases, the ambulatory medical device is configured to operate without input from the patient. Some ambulatory medical devices are configured to administer medicament responsive to received input from the patient. Many ambulatory medical devices are configured to administer medicament to the patient away from medical supervision. There is a continual need in the art for improvements in ambulatory medical devices to enhance all aspects of their operation.

### DETAILED DESCRIPTIONS OF THE INVENTIONS

The present inventors have discovered novel ways to use GUIs, widgets and other screen-based items along with new patch pump configurations without substantial negative impacts on cognitive load.

The disclosed subject matter is a system for delivering medicaments using an ambulatory medical device. The system determines the amount of medicament to deliver to a user based on input and feedback from the user. In various embodiments, the system may determine an amount of medicament based on biomarker feedback from the user. For example, various algorithms may be used to determine the amount of medicament to deliver to the user.

The amount of medicament may be varied continuously and dynamically over time. Likewise expressly incorporated herein by reference are issued [U.S. Pat. Nos. 11,594,313; 11,688,501; 8,273,052; 9,833,570 and 11,610,661; 12,204,889; 12,115,388; 12,093,681] and pending patch pump cases including U.S. Ser. No. 19/183,101 and PCT/US25/25374, all intellectual properties of the instant assignee Beta Bionics, Inc. (NYSE: BBNX, Irvine, CA, USA) or expressly under license.

In an exemplary embodiment, the medicament may include insulin. In another exemplary embodiment, the medicament may include glucagon. Insulin is a hormone that regulates blood glucose levels by facilitating glucose uptake into cells. Glucagon is a hormone that raises blood glucose levels by promoting the release of stored glucose from the liver. In various embodiments, the medicament may be delivered automatically by the ambulatory medical device. The term ambulatory, as used herein, describes a device that may be worn by the user and is not connected to any stationary device such as connected to a wall or outlet. The ambulatory medical device allows the user to freely travel and go where they please while the ambulatory medical device treats the patient or user. The ambulatory device may be carried by the user or may be adhered to the patient's body.

In various embodiments, the ambulatory medical device may include an insulin sensitivity factor. The insulin sensitivity factor may be determined using various algorithms and may be adjusted continuously over time. The insulin sensitivity factor, as used herein, may include a gain term that determines the aggressiveness with which the patient is treated with insulin. In various embodiments, the gain term may also be applied to other medicaments besides insulin. In an exemplary embodiment, the ambulatory medical device may determine an amount of unmetabolized insulin in the user. In yet another exemplary embodiment, the ambulatory medical device may determine a basal rate in the user. The basal rate, as used herein, may refer to the rate at which insulin is produced by the user. In yet another exemplary embodiment, the ambulatory medical device may include one or more meal adaptations. For example, the ambulatory medical device may learn to adjust medicament delivery based on meals taken by the user.

The ambulatory medical device may connect to a user to deliver medicament to the user. For example, the ambulatory medical device may be worn by the user administer medicament using a transdermal needle. The ambulatory medical device may include a pump that delivers medicament to the user. In an embodiment, the pump is a lead screw-driven syringe pump that includes a motor connected to a lead screw that converts rotational motion into linear motion. The lead screw may drive a plunger within a syringe to dispense a controlled amount of medicament to a user at a predetermined rate. Another example of a pump may be a peristaltic pump, which moves fluid through a flexible tube using rotating rollers. The ambulatory medical device may further include a controller that determines an amount of medicament and instructs the pump to deliver the medicament to the user. The ambulatory medical device may also include one or more reservoirs for medicament. The controller may determine an amount of medicament to deliver to the user. The ambulatory medical device may continuously modify or adjust the amount of medicament based on time and various sensor inputs from the user. For example, the ambulatory medical device may collect biomarker data from the user and adjust the amount of medicament to be delivered. The ambulatory medical device may also be operated by the user, such as allowing the user to enter mealtimes or meal breaks and specify the size of a meal.

An exemplary embodiment of the ambulatory medical device includes a corrections controller. The corrections controller may be a control mechanism that calculates small insulin doses based on near-continuous calculations of the deviation of current glucose from a target level. The controller may include a gain term that controls how many units of insulin to deliver per mg/dL that the glucose is above target. The gain term can be considered an insulin sensitivity factor or an aggressiveness term. Additionally, the controller may take into account any insulin that has been delivered but has not yet been metabolized. The insulin amount that has not yet been metabolized is the insulin on board.

The gain term of the ambulatory medical device controls an amount of medicament delivery. For example, the gain term in an insulin-delivering medical device may adjust the insulin sensitivity factor, which determines the amount of medicament delivered per time period to a user. A higher gain term may result in more aggressive medicament delivery, while a lower gain term may result in less aggressive medicament delivery. The gain term may change over time. The ambulatory medical device may collect biomarker data and adapt the gain term based on the biomarker data. For example, the gain term may be modified by the ambulatory medical device based on glucose readings from the user. The ambulatory medical device may vary the gain term based on each user. Further, the gain term may be adjusted based on past delivery data.

The gain term may be initialized based on various parameters such as body mass, total daily dose, and similar factors. In an exemplary embodiment, the gain term may be adapted based on glucose statistics. For example, a target glucose statistic may be determined by evaluating glucose levels at regular time intervals. If glucose at the end of a time period is greater than a conditional threshold, the gain term may be increased by a set percentage. If glucose at the end of the time period is below a conditional threshold, the gain term may be decreased by a set percentage. For example, if the total time that the glucose is below 70 mg/dL over the past 24 hours exceeds 5% then the gain term may be decreased, or if the total time that the glucose is below 120 mg/dL is less than 5% it may be increased

Similarly, the gain term may be adjusted based on mean glucose levels. If the mean glucose is greater than a target, such as 150 mg/dL, the gain term may be increased. If the mean glucose is less than the target, the gain term may be decreased. Other statistical methods may also be used to adjust the gain term. For example, both the mean and standard deviation of glucose levels may be used to calculate a lower glucose limit. The lower limit may be determined using the measured mean, standard deviation, and a t-statistic with a significance level of 0.10. If the calculated lower statistic is below a threshold, the gain term may be decreased. If the calculated lower statistic is above the threshold, the gain term may be increased.

The gain term may also be adjusted based on the statistics of medicament delivery. For example, a patient may have a specific total daily insulin dose that is expected, and the gain term may be increased if the actual insulin delivered is below this target. Similarly, in a bi-hormonal system using insulin and glucagon, the insulin gain term may be decreased when glucagon dosing is higher than expected.

The gain term may also be adjusted when glucose is very high. For instance, the gain term may be reduced to prevent abnormally high insulin doses. In an example of use, a system may linearly decrease the gain term with glucose levels starting with full ‘gain term’ at 250 mg/dL and decreasing to half ‘gain term’ at 400 mg/dL.

In an exemplary embodiment, the ambulatory medical device may determine insulin on board and adapt various medicament delivery amounts based on the insulin on board. The insulin on board, as used herein, refers to unmetabolized insulin in the patient. When a patient starts it is unclear what their insulin on board is. If the patient's glucose is high, they may also have high insulin on board to bring that glucose down that was delivered from a previous pump or pen injection. If glucose is high and insulin on board is high and if the system does not know about the insulin on board, the system may over-deliver insulin. On start-up, a system may calculate the amount of insulin needed for the current glucose levels and assume that there is that much insulin on board which will reduce the initial corrections dosing until any residual insulin on board is metabolized.

Every patient or user metabolizes insulin at a different rate. Accordingly, the ambulatory medical device may adjust the determination of insulin on board specifically for each user. A determination of the remaining un-metabolized insulin in the patient may be performed using an estimate of insulin t-max, which is the time at which the pharmacodynamic effect of a single insulin dose reaches its maximum level. Alternatively, the calculation may use insulin duration, which represents the total period during which a single dose of insulin affects the patient's glucose levels. These insulin activity parameters may vary from patient to patient. The system may adapt these parameters to the individual patient.

The ambulatory medical device may also factor in a basal rate for the user. Each patient may have a basal rate of endogenous glucose production, which ideally may be compensated for by the basal insulin rate. The nominal basal rate can be estimated and adapted, with instantaneous adjustments made based on glucose levels. For example, a sensor may receive glucose readings from the user and transmit them to the controller. Based on the glucose levels, a basal rate can be estimated. In various embodiments, an initial basal rate may be estimated based on user parameters such as body mass and total daily dose.

In an exemplary embodiment, a nominal basal rate may be adjusted using a method similar to the insulin sensitivity factor adaptation referenced above. For example, glucose levels may be evaluated over a 24-hour period, with readings collected every six hours. Each time a glucose level statistic violates a threshold, the basal rate may be decreased by a certain percentage. Each time a glucose level statistic violates a different threshold, the basal rate may be increased by a certain percentage. For example, if the total time that the glucose is below 70 mg/dL over the past 24 hours exceeds 5% then the basal rate may be decreased, or if the total time that the glucose is below 120 mg/dL is less than 5% it may be increased.

In an exemplary embodiment, the basal rate may be determined based on the amount of medicament previously delivered. For example, a nominal basal rate may represent the amount of insulin required to counteract a patient's nominal endogenous glucose production (EGP). The EGP rate may correspond to the lowest amount of insulin delivered throughout the day.

Accordingly, the nominal basal rate may be adjusted by monitoring insulin delivery over time. For instance, the basal rate may be set based on insulin delivery during a time period with the lowest insulin delivery, the basal rate may then be further adjusted based on changes in insulin on board and glucose levels during that time period, setting the basal rate to the nominal basal rate.

The adjustments for insulin on board (IOB) may be made by subtracting the IOB at the beginning of the period from the IOB at the end of the period and using this difference to adjust the measured insulin delivery. Similarly, if glucose levels dropped during the period, the measured insulin delivery may be decreased, as it indicates that insulin delivery exceeded the patient's endogenous glucose production. Conversely, if glucose levels increased, the measured insulin delivery may increase.

In embodiments, the system may undo learning when an occlusion is detected. For example, if an occlusion is detected, look back for a period of time and undo learning of the nominal basal rate and the gain term. The look-back period may be fixed or may be based on insulin delivery. For example, occlusions may require 3 units of insulin to detect. Accordingly, the system may look back for the last 3 units of insulin or monitor motor current history to determine when the current increased.

In an exemplary embodiment, the ambulatory medical device includes a meal controller. The meal controller may adjust medicament delivery based on meals taken by the user. For example, a user may use one or more inputs on the ambulatory medical device to indicate that a meal is being taken. The ambulatory medical device may then adjust medicament delivery based on this input. In various embodiments, the ambulatory medical device may learn from past meals and biomarker feedback to adjust the amount of medicament or insulin delivered in response to a meal. For example, if a user exhibits a consistent biomarker reaction to a specific meal over time, the ambulatory medical device may adjust insulin delivery near mealtime based on the user's historical biomarker data.

In embodiments of the disclosed subject matter, the system may determine a time at which a medicament reaches a maximum concentration in the subject. The time at which the medicament reaches the maximum concentration may be referred to as t-max. Based on t-max, the amount of unmetabolized medicament may be determined. Based on the unmetabolized medicament, additional medicament administration may be determined as well.

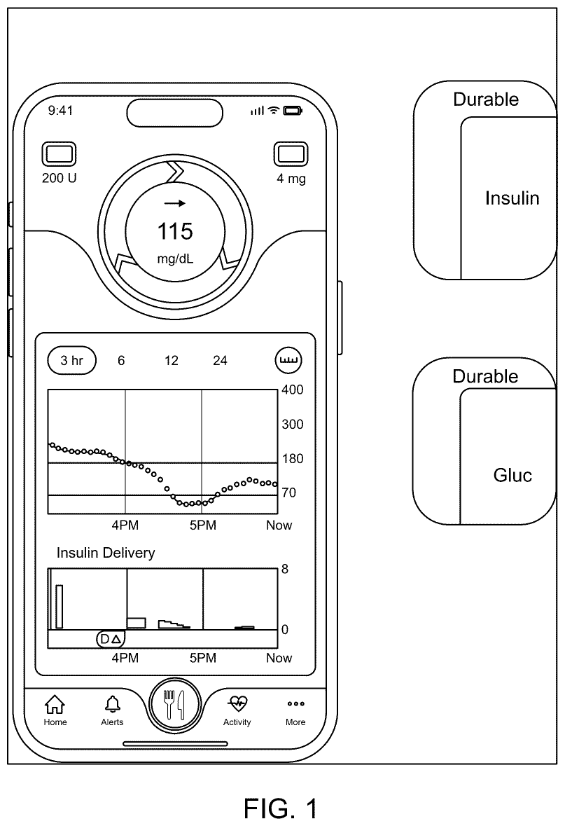

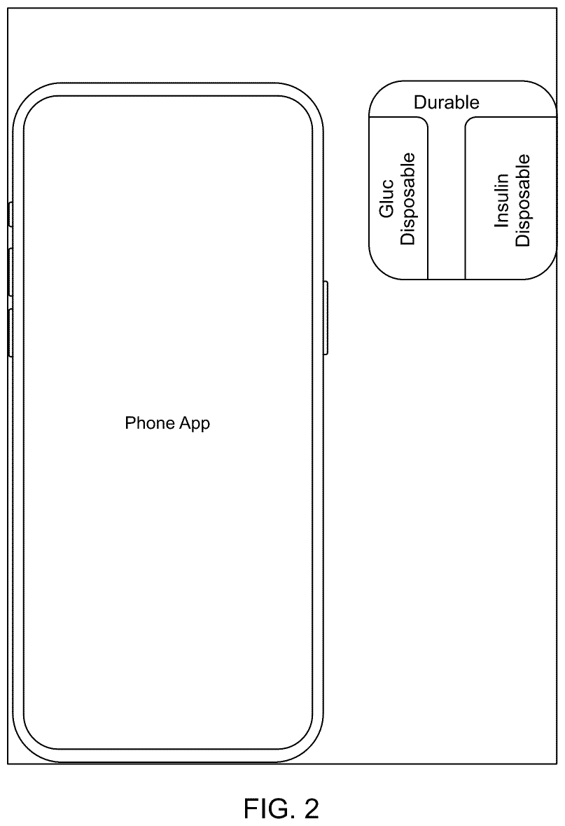

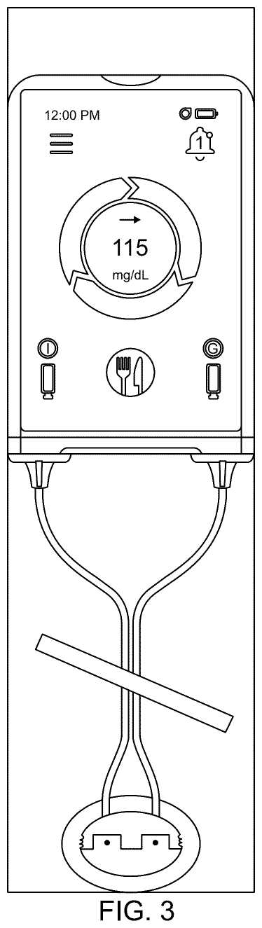

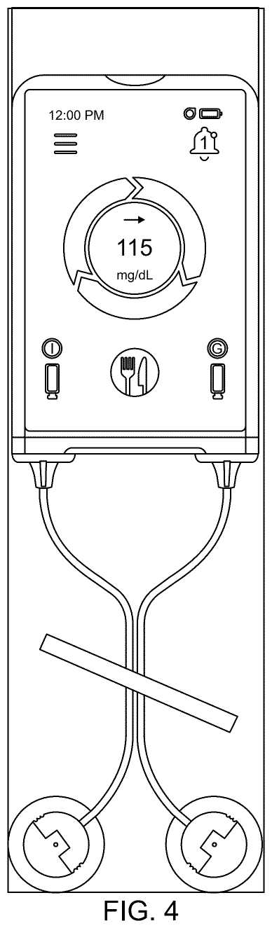

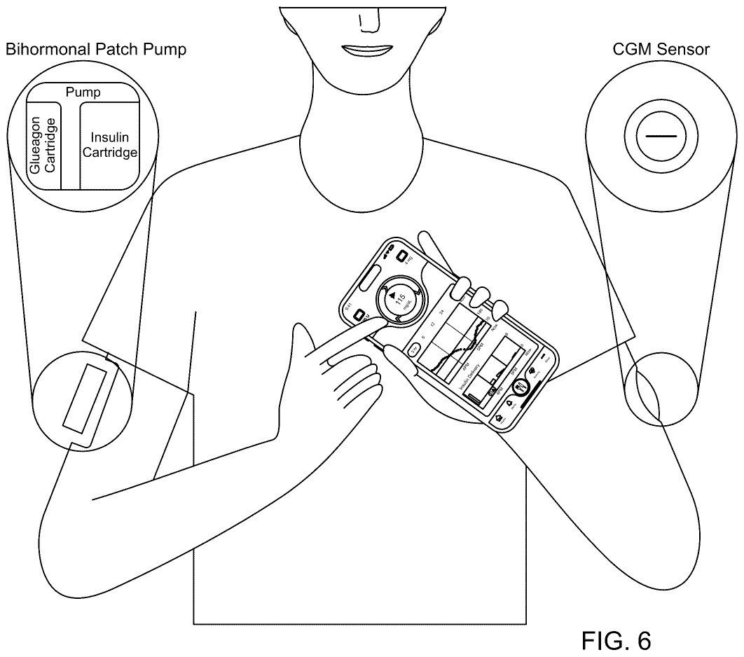

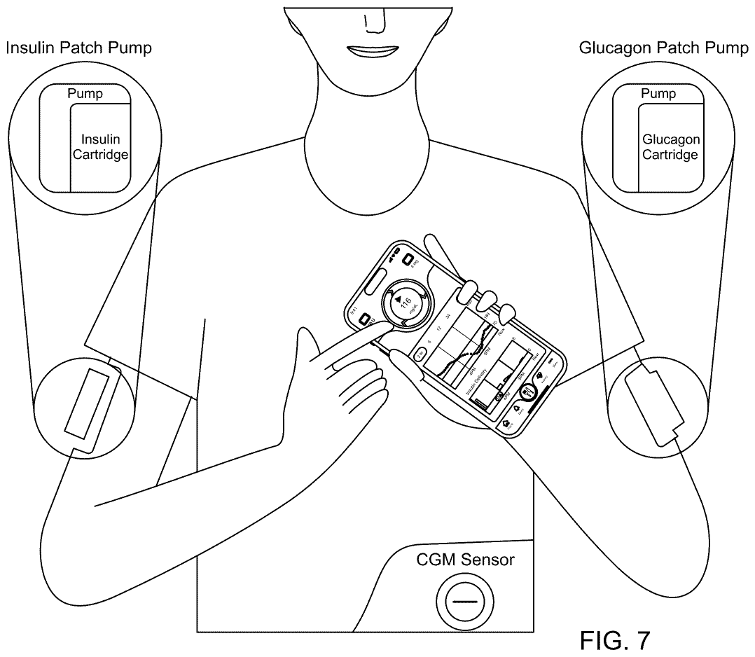

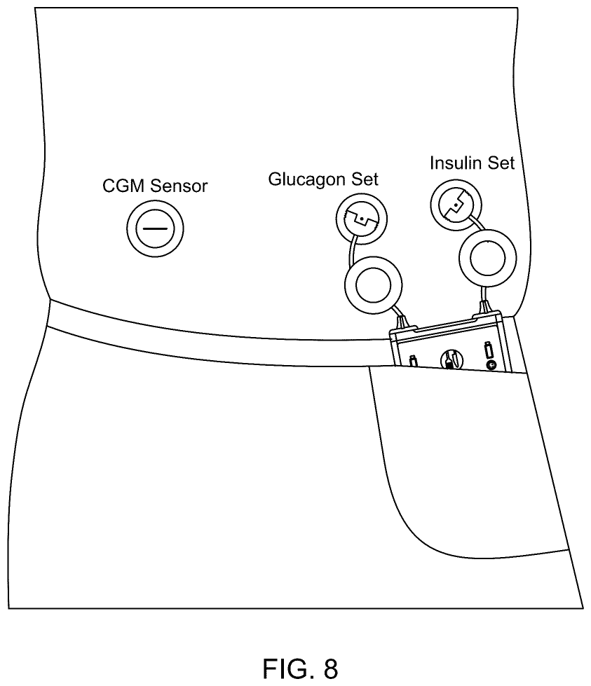

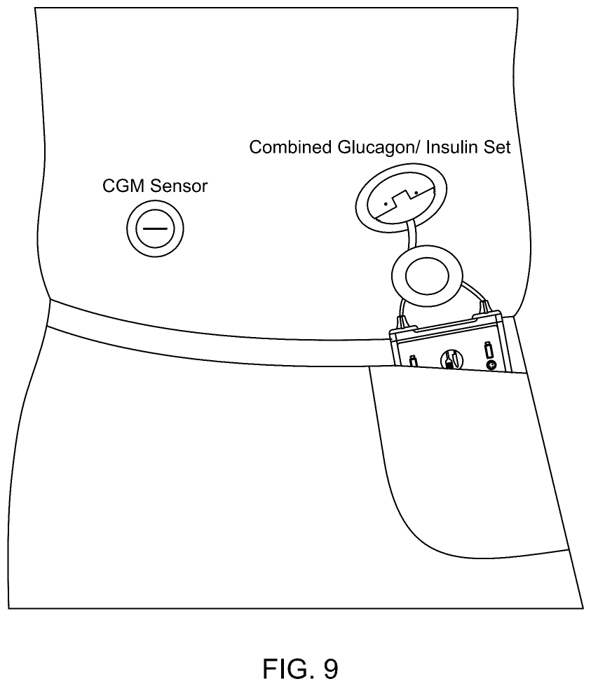

Referring to FIG. 1, FIG. 1 through 9 are integrated herein as previously disclosed and further explained in co-pending USSN at the USPTO, along with all of the table bound and listed patent and patent applications of Applicant from provisional patent applications associated herewith. In short, according to the instant teachings systems were patented from at least as early as December of 2020 for autonomous systems functioning based solely upon a priming dose associated with user's bodyweight with a learning algorithm on a patch.

Patch Pumps'transmission of adapted parameters is controlled by various triggers SEE for example BETA BIONICS′ U.S. Pat. Nos. 11,594,313; 11,688,501 and 11,610,661. User interactions keep patches up to date and infusion algorithms constantly improving. FIG. 1-3 depicting myriad options such as 2 patches 2 cannulae to Boolean aggregates therefrom based in part on a first option being 2 sites, 2 cannulae and steel or Teflon/PTFE.

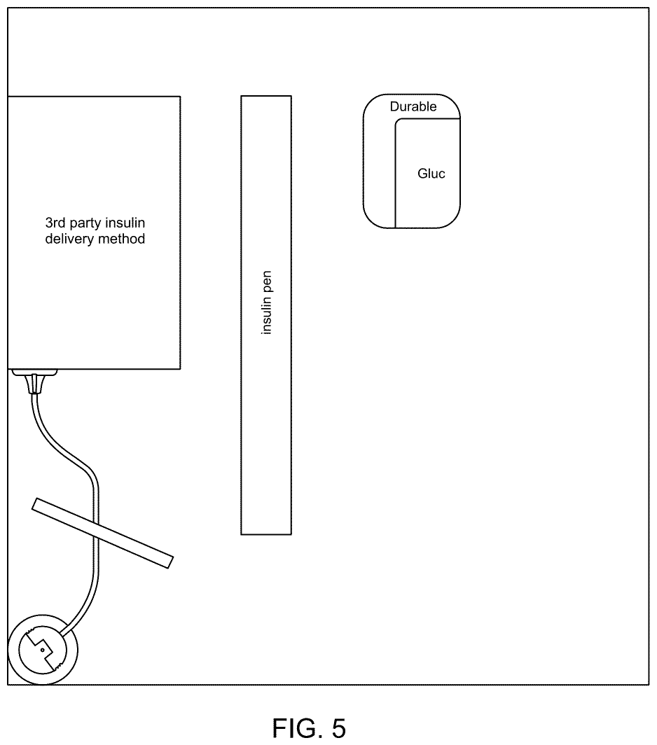

FIG. 5 through FIG. 9 highlight and underscore aspects of the instant teachings as applied to a novelty-based method for servicing third party insulin delivery methods. Artisans are well aware that each of the prior combinations may be used to address this approach, as explained and claimed below.

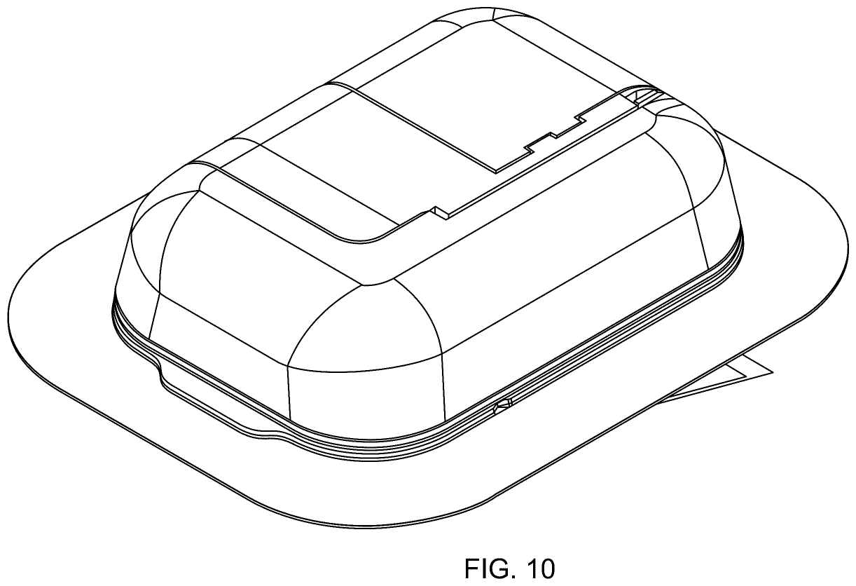

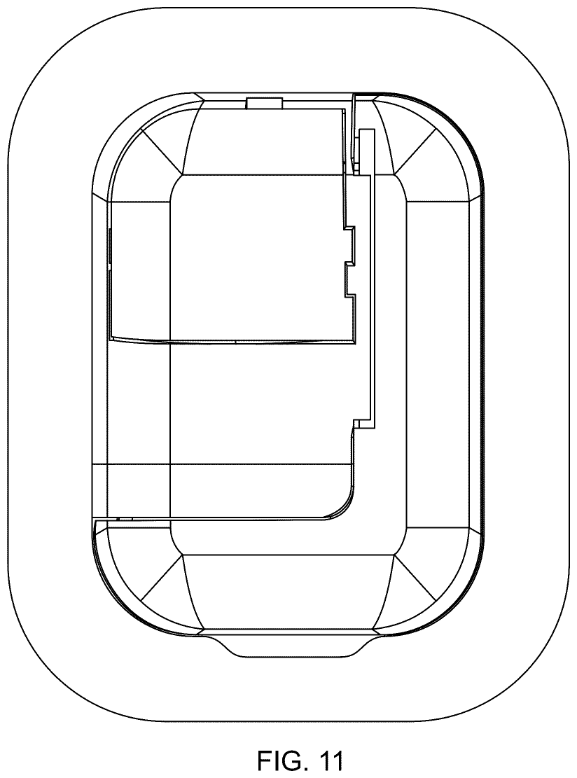

Referring now to FIG. 10 and FIG. 11 Patch Pump systems (PPs) are defined herein as at least one of the iLET BIONIC PANCREAS Patch Pump system, the iLET BIONIC PANCREAS Patch Pump Bihormonal system and the iLET BIONIC PANCREAS Patch Pump system, a tubeless ACEpump, there are shown a bottom adhesive section for housing at least one of a steel cannulae, Teflon (PTFE) cannulae, micro-needle arrays to provide ingress to the patient/user. As is known, these new infusion pumps can be small, light-weight and easy to use. Such Patch pumps stand as improvements in certain clinical settings, but have enumerated strong needs to improve fluid driving mechanisms from reservoirs through transcutaneous access tools-the present inventors hereby offer for consideration one set of such systems, as expressly labeled as desiderata by industry, including over the INSULET OMNIPOD brand of device (see U.S. Pat. No. 10,420,883 Column 2, line 2).

### Examples One and Two the Patch Pump Inventions

Beta Bionics have discovered that closed-loop systems for treating diabetes are manifested in patch pumps both in combination with extant BETA BIONICS systems (as referenced above) and in combination with third party insulin delivery methods.

In essence, there a several principled approaches comprising combinations of novel drive train concepts with glucagon pumps which respond to glucose level control signals, inter alia.

For example, the Beta Bionics Bihormonal system using the iLET App for the Bionic Pancreas Patch Pump, and standalone systems working as accessories to third party insulin delivery systems are disclosed here and claimed below.

The iLET Bionic Pancreas Patch Pump, and standalone systems working as accessories to third party insulin delivery systems are disclosed and claimed herein. FIGS. 5-8 describe these embodiments to those skilled in the art and constitute progress in science and the useful art.

Novelties of the iLET App for the Bionic Pancreas Patch Pump, and standalone systems working as accessories to third party insulin delivery systems are disclosed which leverage the drivetrain embodiments reduced to practice for these systems comprised of single motor to drive two medicaments, one device (clutch, ratchet); Nitinol wire embodiments to drive two channels and multi-medicament systems; and chimeric disclosures including those is closed in co-pending 63/627,278 of BETA BIONICS expressly incorporated herein by reference as if full set forth herein.

### EXAMPLE

### Modular Use of Patch Pump Set-Up

Patch pump with adaptive learning is still initially set up according to the same communications interface and of the glucose control system inventions disclosed herein.
As disclosed in co-pending U.S. Ser. No. 19/183,101—and PCT/US25/25374—Durable and disposable components connected together;
Primed with body weight only
No further training required. Those skilled in the art understand that this means each feature set selected for and paired from the App can be seamlessly transferred from, to and vise-versa for each components or feature.

According to embodiments there is shown a method for administering medicament from at least a patch pump medical device, the method comprising:

Merely connecting the disposable and durable components in operative contact with a user;
determining an amount of medicament to administer to a user based on the body weight of a user;
computing the amount of medicament to administer to the user; and adjusting the amount of medicament to transfer to the user based on the computation;
transmitting at least said instruction to the patch pump medical device to deliver the amount of medicament to the user.
   The method for administering medicament from at least a patch pump medical device of claim 18, being further comprised of, in combination:
a meal announcement slider associated with a time period;
a fractional depiction of breakfast, lunch and dinner; and
at least a means for changing the fractional amount of the associated meal.

The method for administering medicament from at least a patch pump medical device of claim 19, wherein the fractional depiction is adjusted for at least two periods of time wherein the time period is defined by 10 PM to 4 AM and a second time period for as the carbohydrate slider allows a predetermined meal to be changed from one percentage of usual, whereby using GUI adjustments are made to the settings which are then delivered.

### EXAMPLE

### Modular Use of Patch Pump Setting With Biomarker Adjustments

A method for administering medicament from a medical device includes receiving, from a sensor, a first biomarker level in a user, receiving a time of peak pharmacodynamic effect (t-max) in the user, determining an amount of unmetabolized medicament in the user based on the t-max, determining an amount of medicament to administer to the user based on the amount of unmetabolized medicament in the user, transmitting a first instruction to the medical device to deliver the amount of medicament to the user, further receiving, from the sensor, a second biomarker level in the user, updating the t-max based on the second biomarker level, updating the amount of unmetabolized medicament in the user based on the updated t-max, adjusting the amount of medicament to transfer to the user based on the adjusted t-max, and transmitting a second instruction to the medical device to deliver the amount of medicament to the user.

The method may include initiating determination of the t-max based on a condition. The second biomarker level may be a concentration, and the condition may be triggered by the concentration exceeding a value. Updating the t-max may include determining amounts of unmetabolized medicament over a period of time for multiple t-max values and comparing the amounts of unmetabolized medicament to the second biomarker level over the period of time. Determining the t-max may be initiated based on the condition and a meal announcement. The method may include determining a basal rate in the user, and updating the amount of unmetabolized medicament may include factoring the basal rate. The method may also include adjusting the basal rate based on the first biomarker level and the second biomarker level.

A system for determining an amount of unmetabolized medicament in a user includes a sensor configured to measure a biomarker level in the user and a controller. The controller is configured to receive a first biomarker level from the sensor, receive a time of peak pharmacodynamic effect (t-max), determine an amount of unmetabolized medicament in the user based on the t-max, determine an amount of medicament to administer to the user based on the amount of unmetabolized medicament, transmit a first instruction to a medicament pump to administer the determined amount of medicament, receive a second biomarker level from the sensor subsequent to administration of the medicament, update the t-max based on the second biomarker level, update the determined amount of unmetabolized medicament based on the updated t-max, adjust the amount of medicament administered to the user based on the updated amount of unmetabolized medicament, and transmit a second instruction to the medicament pump to deliver the adjusted amount of medicament. The controller may be configured to initiate determination of the t-max based on detecting at least one condition from the first biomarker level. The biomarker level may be a glucose concentration, and the condition may include the glucose concentration exceeding a predetermined threshold. The controller may determine multiple theoretical amounts of unmetabolized medicament using multiple t-max values and may select the updated t-max based on comparing each theoretical amount with the second biomarker level. The condition may further include receiving a meal announcement from the user. The controller may determine a basal rate of endogenous medicament production in the user, and updating the amount of unmetabolized medicament may include adjusting based on the basal rate. The controller may adjust the basal rate based on statistical analysis of biomarker levels collected over time, including a mean or standard deviation of glucose concentration.

An ambulatory medical device configured to administer insulin to a user includes a reservoir configured to store insulin, a pump configured to deliver insulin from the reservoir to the user, a sensor configured to measure glucose concentration levels in the user, and a controller. The controller is configured to receive a first glucose concentration level from the sensor, determine an initial t-max indicating a time of peak insulin effect in the user, determine an amount of unmetabolized insulin in the user based on the t-max, determine an amount of insulin to deliver based on the amount of unmetabolized insulin, control the pump to deliver the determined amount of insulin, receive a second glucose concentration level subsequent to insulin delivery, update the t-max based on a comparison between the second glucose concentration level and multiple theoretical insulin activity curves calculated from different t-max values, and adjust the amount of insulin delivered based on the updated t-max. The controller may be configured to initiate determining the t-max upon identifying at least one triggering condition in the first glucose concentration level. The condition may include the glucose concentration exceeding a threshold, decreasing consecutively for a predetermined number of measurements, or decreasing below a defined threshold within a defined time period. The controller may be configured to continuously refine the t-max value by iteratively comparing theoretical insulin activity curves based on updated t-max values against subsequently measured glucose concentrations. The controller may refine the t-max based on a user's historical glucose responses at different times of day or in response to meal consumption. The controller may also prevent insulin overdosing by initializing the amount of unmetabolized insulin as substantially equal to a calculated insulin requirement derived from an initial measured glucose concentration in the absence of historical insulin data upon device start-up.

Many variations may be made to the embodiments described herein. All variations, including combinations of embodiments, are intended to be included within the scope of this disclosure. The description of the embodiments herein can be practiced in many ways. Any terminology used herein should not be construed as restricting the features or aspects of the disclosed subject matter. The scope should instead be construed in accordance with the appended claims.

1. A patch pump durable and disposable system comprised of, in combination:
at least a drive coupling and sensing mechanisms;
methodology, comprising, in combination;
at least a one way drive coupling and fill detection apparatus; and
travel to interface with power management for both disposable and durable options including a pump for use with delivery of therapy for diabetes.

2. The patch pump durable and disposable system of claim 1, used with at least a first drive nut having a length extending longitudinally along the pump housing, the first drive nut extending into the first medicament cartridge receptacle via the first aperture and in communication with the first lead screw, the first drive nut being configured to urge forward or backward in response to a first direction of rotation and a second direction of rotation, respectively, of the first lead screw.

3. The patch pump durable and disposable system of claim 2, whereby once the nut is past a point it becomes locked into the rotation system, and a tooth engages the nut and stops it from locking.

4. The patch pump durable and disposable system of claim 3, further comprising the plunger staying until commanded, causing travel to the interface.

5. The patch pump durable and disposable system of claim 4, further comprising so determining at least one of the insulin fill, glucagon fill, and glucagon-like moiety capable of increased shelf and storage-life DMSO fill.

6. The patch pump durable and disposable system of claim 5, not using a clutch type of mechanism, functionalized by one stop assembly, which is comprised of attachment of the durable and disposable components and only priming with body weight of a user.

7. The patch pump durable and disposable system of claim 6, further comprising a first seal, the first seal providing a water resistant barrier between the internal area of the infusion pump and the first medicament cartridge receptacle; wherein the first seal is a first O-ring that surrounds at least a portion of the first drive nut, wherein the pump housing comprises a first saddle, the first saddle being configured to retain the first O-ring.

8. The patch pump durable and disposable system of claim 7, wherein the first drive nut comprises a at least a tooth/protrusion and the pump housing comprises a groove configured to receive the protrusion, where the tooth/ protrusion engages the groove and prevents rotation of the drive.

9. A fluid delivery system comprising, in combination: at least a fluid reservoir, for having a transcutaneous access tool fluidly coupled thereto; a transcutaneous access tool fluidly coupled to the fluid reservoir; and an improved drive mechanism for drive coupling and fill detection.

10. The fluid delivery system of claim 9, being at least a patch pump and remote control with adaptive learning means comprised of a system which further comprises in combination: primary; secondary and tertiary means for dosing and delivering medicaments to patients.

11. The fluid delivery system of claim 10, being a collection of disposable and durable wearable medical devices or patch pump kit.

12. The fluid delivery system of claim 11, being comprised of at least an infusion set and: 2 patches, 2 cannulas.

13. The fluid delivery system of claim 11, being comprised of at least: 1 patch, 2 cannulas.

14. The fluid delivery system of claim 11, being comprised of 1 site, 2 cannulas, steel only.

15. The fluid delivery system of claim 11, being comprised of 2 sites, 2 cannulas steel or Teflon.

16. The fluid delivery system of claim 11, being comprised of glucagon patch control governed in, at least one of the following configurations:
standalone glucagon pump & 3rd party insulin delivery.

17. The fluid delivery system of claim 11, being comprised of at least one of:
2 patches, 2 cannulas;.
1 patch, 2 cannulas;
1 site, 2 cannulas, steel only; and
2 sites, 2 cannulas steel or Teflon.

18. A method for administering medicament from at least a patch pump medical device, the method comprising:
merely connecting the disposable and durable components in operative contact with a user;
determining an amount of medicament to administer to a user based on the body weight of a user;
computing the amount of medicament to administer to the user;
adjusting the amount of medicament to transfer to the user based on the computation; and
transmitting at least said instruction to the patch pump medical device to deliver the amount of medicament to the user.

19. The method for administering medicament from at least a patch pump medical device of claim 18, being further comprised of, in combination:
a meal announcement slider associated with a time period;
a fractional depiction of breakfast, lunch and dinner;
at least a means for changing the fractional amount of the associated meal; and
wherein the fractional depiction is adjusted for at least two periods of time wherein the time period is defined by 10 PM to 4 AM and a second time period for as the carbohydrate slider allows a predetermined meal to be changed from one percentage of usual, whereby using GUI adjustments are made to the settings which are then delivered.
